(19) United States
(12) Patent Application Publication
(10) Pub. No.: US 2025/0186237 A1
(43) Pub. Date: Jun. 12, 2025

(54) CURVED LINKAGE SYSTEM FOR A SHOULDER BRACE

(71) Applicant: Breg, Inc., Carlsbad, CA (US)

(72) Inventor: Gary Bledsoe, Mansfield, TX (US)

(21) Appl. No.: 18/958,154

(22) Filed: Nov. 25, 2024

Related U.S. Application Data
(60) Provisional application No. 63/607,813, filed on Dec. 8, 2023.

(51) Int. Cl. A61F 5/01

(52) U.S. Cl. CPC A61F 5/013; A61F 2005/0167

(57) ABSTRACT

The present disclosure relates to a curved linkage system for connecting an arm support to a waist plate of a shoulder brace, the curved linkage system comprising two curved link pairs that each comprise an upper curved link and a lower curved link that each comprise a proximal cylindrical joint and a distal cylindrical joint connected by an elongated body, wherein the two curved links of each curved link pair are positioned in parallel so the proximal cylindrical joints, the elongated bodies, and the distal cylindrical joints are each aligned with each other, the first curved link of the first curved link pair is configured to connect to a support lug protruding from the waist plate through the proximal cylindrical joints, the proximal cylindrical joints of the second curved link pair fit inside the distal cylindrical joints of the first curved link pair when connected.

### CROSS-REFERENCE SECTION

This application claims priority to U.S. Provisional Application No. 63/607,813, filed on Dec. 8, 2023, which is incorporated by reference herein in its entirety for all purposes.

### FIELD OF THE DISCLOSURE

The present disclosure relates, according to some embodiments, to a curved linkage system that assists in increasing limb range-of-motion for non-surgical or post-operative stabilization orthopedic shoulder braces.

### BACKGROUND

Shoulder stiffness is often a result of inflammation of the capsule located in the shoulder joint, which causes significant pain and loss of passive range of motion. An example shoulder stiffness condition is adhesive capsulitis, which affects from 2-6% of the general population, including about 20% of diabetic patients. Additionally, one of the most common causes of shoulder stiffness is shoulder surgery, such as from arthroscopic or open surgery, rotator cuff repair, instability surgery, decompressions, labral repairs, fracture surgery, and shoulder arthroplasty. Many surgeons immobilize the shoulder of a patient after surgery to protect the repair, leading to stiffness. Osteoarthritis, secondary injuries to surrounding tissues, and strokes are yet other causes of shoulder stiffness.

Treating shoulder stiffness generally includes mobility rehabilitation, including stretching exercises that have to be done multiple times a day for them to be effective. Further, as treatment plans progress, so do the range and duration of the stretching involved in the mobility rehabilitation. Known shoulder braces immobilize the shoulder joint and are thus limited to a static function. To move the shoulder joint requires taking off known shoulder braces. What is needed is a shoulder brace having both a static function and an additional repositioning function that permits dynamic stretching.

### SUMMARY

The present disclosure relates to a curved linkage system for connecting an arm support to a waist plate of a shoulder brace, the curved linkage system including a first curved link pair including an upper curved link and a lower curved link that each include a proximal cylindrical joint and a distal cylindrical joint connected by an elongated bode. The two curved links may be positioned in parallel so the proximal cylindrical joints, the elongated bodies, and the distal cylindrical joints may be each aligned with each other. The first curved linked may be configured to connect to a support lug protruding from the waist plate through the proximal cylindrical joints. The distal cylindrical joints of the first curved link pair may be configured to connect to proximal cylindrical joints of a second curved link pair. The curved linkage system may include a second curved link pair including an upper curved link and a lower curved link that each include a proximal cylindrical joint and a distal cylindrical joint connected by an elongated body. The two curved links may be positioned in parallel so that the proximal cylindrical joints, the elongated bodies, and the distal cylindrical joints may be each aligned with each other. The proximal cylindrical joints of the second curved link pair may fit inside the distal cylindrical joints of the first curved link pair when connected. The distal joints of the second curved link pair may be configured to connect to a pivot post protruding from a bottom surface of the arm support.

The curved linkage system may include a knob having a throughbolt that inserts and threadably screws into a pivot post, the knob configured to adjust a level of incremental rotational adjustment of an arm support with respect to a curved linkage. The curved linkage system may further include a throughbolt that passes through part of the first curved linked and a vertical axial hole of a support lug; and a nut fastened to an end of the throughbolt to secure a first curved link to the support lug. The curved linkage system may include at least one of a serrated disk and a friction washer fitted in between a support lug and the first curved link. The curved linkage system may include a tension knob having a throughbolt configured to secure the distal joint of the second curved link pair to the pivot post. In some embodiments, each of the first curved link pair and the second curved link pair provides for an about 12 inch radius of movement. The first curved link pair may be made from a metal including steel, iron, aluminum, titanium, zinc, bronze, chromium, tin, tungsten, zinc, and alloys thereof. The second curved link pair may be made from a metal including steel, iron, aluminum, titanium, zinc, bronze, chromium, tin, tungsten, zinc, and alloys thereof. The first curved link pair may be made from a polymer including polyethylene, polystyrene, nylon, polypropylene, polyvinylchloride, neoprene, cellulose, rubber, silicone, tetrafluoroethylene, polyamide, polybutadiene, and copolymers thereof. The second curved link pair may be made from a polymer including polyethylene, polystyrene, nylon, polypropylene, polyvinylchloride, neoprene, cellulose, rubber, silicone, tetrafluoroethylene, polyamide, polybutadiene, and copolymers thereof.

A curved linkage system for connecting an arm support to a waist plate of a shoulder brace, the curved linkage system including a first curved link pair including an upper curved link and a lower curved link that each include a proximal cylindrical joint and a distal cylindrical joint connected by an elongated bode. The two curved links may be positioned in parallel so the proximal cylindrical joints, the elongated bodies, and the distal cylindrical joints may be each aligned with each other. The first curved linked may be configured to connect to a support lug protruding from the waist plate through the proximal cylindrical joints. The distal cylindrical joints of the first curved link pair may be configured to connect to proximal cylindrical joints of a second curved link pair. The curved linkage system may include a second curved link pair including an upper curved link and a lower curved link that each include a proximal cylindrical joint and a distal cylindrical joint connected by an elongated body. The two curved links may be positioned in parallel so that the proximal cylindrical joints, the elongated bodies, and the distal cylindrical joints may be each aligned with each other. The proximal cylindrical joints of the second curved link pair may fit inside the distal cylindrical joints of the first curved link pair when connected. The curved linkage system may include a third curved link pair including an upper curved link and a lower curved link that each include a proximal cylindrical joint and a distal cylindrical joint connected by an elongated body. The two curved links may be positioned in parallel so that the proximal cylindrical joints, the elongated bodies, and the distal cylindrical joints may be each aligned with each other. The proximal cylindrical joints of the second curved link pair may fit inside the distal cylindrical joints of the second curved link pair when connected. The curved linkage system may include a fourth curved link pair including an upper curved link and a lower curved link that each include a proximal cylindrical joint and a distal cylindrical joint connected by an elongated body. The two curved links may be positioned in parallel so that the proximal cylindrical joints, the elongated bodies, and the distal cylindrical joints may be each aligned with each other. The proximal cylindrical joints of the second curved link pair may fit inside the distal cylindrical joints of the third curved link pair when connected. Distal joints of the fourth curved link pair may be configured to connect to a pivot post protruding from a bottom surface of the arm support.

### BRIEF DESCRIPTION OF THE DRAWINGS

Some embodiments of the disclosure may be understood by referring, in part, to the present disclosure and the accompanying drawings, wherein:

FIG. 1 illustrates a top view of a disclosed curved linkage system connecting an arm support to a waist plate of a shoulder brace, according to an example embodiment of the disclosure;

FIG. 2 illustrates the right front oblique view of the curved linkage system of FIG. 1, according to an example embodiment of the disclosure;

FIG. 3 illustrates a rear side view of the curved linkage system of FIG. 1, according to an example embodiment of the disclosure;

FIG. 4 illustrates a front view of the curved linkage system of FIG. 1, according to an example embodiment of the disclosure;

FIG. 5 illustrates a rear top view of a disclosed curved linkage system having two curved link pairs, according to an example embodiment of the disclosure;

FIG. 6 illustrates a front view of the curved linkage system of FIG. 5, according to an example embodiment of the disclosure;

FIG. 7 illustrates rear view of the curved linkage system of FIG. 5, according to an example embodiment of the disclosure;

FIG. 8 illustrates a front side view of the curved linkage system of FIG. 5, according to an example embodiment of the disclosure;

FIG. 9 illustrates two side views of the curved linkage system of FIG. 5 in various positions, according to an example embodiment of the disclosure;

FIG. 10 illustrates a side view of the curved linkage system of FIG. 5 in a different position than those shown in FIG. 9, according to an example embodiment of the disclosure;

FIG. 11 illustrates a top front view of the curved linkage system of FIG. 5, according to an example embodiment of the disclosure;

FIG. 12 illustrates a side view of the curved linkage system of FIG. 5 with the curved link pairs extended outward, according to an example embodiment of the disclosure;

FIG. 13 illustrates a bottom view of the curved linkage system of FIG. 5 with the curved link pairs configured in a 90 degree angle, according to an example embodiment of the disclosure;

FIG. 14 illustrates a top rear view of the curved linkage system of FIG. 5 with the curved link pairs configured in a 90 degree angle, according to an example embodiment of the disclosure;

FIG. 15 illustrates a top rear x-ray view of a curved link pair connecting to a support lug of a waist plate, according to an example embodiment of the disclosure;

FIG. 16 illustrates rear view of the curved linkage system of FIG. 15 with the curved link pairs fully extended, according to an example embodiment of the disclosure;

FIG. 17 illustrates various stages of a user fitted with a shoulder brace having a disclosed curved linkage system, according to an example embodiment of the disclosure;

FIG. 18 illustrates additional adjustment stages of the user fitted with the shoulder brace of FIG. 17, according to an example embodiment of the disclosure;

FIG. 19 illustrates additional adjustment stages of the user fitted with the shoulder brace of FIG. 17, according to an example embodiment of the disclosure;

FIG. 20 illustrates zoomed in views of a reel knob located on a hip support of a disclosed shoulder brace, according to an example embodiment of the disclosure;

FIG. 21 illustrates multiple release mechanisms for removing a disclosed shoulder brace from a user, according to an example embodiment of the disclosure;

FIG. 22 illustrates steps to attach a disclosed shoulder brace to a user, according to an example embodiment of the disclosure;

FIG. 23 illustrates multiple views of a disclosed curved linkage system connecting an arm support to a waist plate of a shoulder brace, according to an example embodiment of the disclosure;

FIG. 24 illustrates multiple views of attaching and detaching curved link pairs from a waist plate, according to an example embodiment of the disclosure;

FIG. 25 illustrates combining two additional curved link pairs to a waist plate attached to two curved link pairs, according to an example embodiment of the disclosure;

FIG. 26 illustrates attaching an arm support to curved link pairs coupled to a waist plate, according to an example embodiment of the disclosure;

FIG. 27A illustrates a waist plate connected to an arm support through four curved links, according to an example embodiment of the disclosure;

FIG. 27B illustrates the waist plate connected to the arm support through the four curved links of FIG. 27A in a different orientation, according to an example embodiment of the disclosure;

FIG. 27C illustrates the waist plate connected to the arm support through the four curved links of FIG. 27A in a different orientation, according to an example embodiment of the disclosure; and

FIG. 27D illustrates the waist plate connected to the arm support through the four curved links of FIG. 27A in a different orientation, according to an example embodiment of the disclosure.

FIGURE COMPONENT TABLE

| | | | | | 1st | 2nd | 3rd | 4th | | | | | | | 4th |
|---|---|---|---|---|---|---|---|---|---|---|---|---|---|---|---|
| | | | | Curved | Curved | Curved | Curved | Curved | | | | | Upper | | Curved |
| | Shoulder | Waist | Arm | Link | Link | Link | Link | Link | | Support | Serrated | Pivot | Curved | Tension | Link |
| Feature | Brace | Plate | Support | system | Pair | Pair | Pair | Pair | Knob | Lug | Disk | Post | Link | Knob | Pair |
| FIG. | 100 | 105 | 110 | 115 | | | | | | 140 | | | | | |
| 1 | | | | | | | | | | | | | | | |
| FIG. | 200 | 205 | 210 | 215 | | | | | 235 | | | 250 | | | |
| 2 | | | | | | | | | | | | | | | |
| FIG. | 300 | 305 | 310 | 315 | | | | | 335 | 340 | | 350 | | | |
| 3 | | | | | | | | | | | | | | | |
| FIG. | 400 | 405 | 410 | 415 | 420 | 425 | | | 435 | 440 | | 450 | | | |
| 4 | | | | | | | | | | | | | | | |
| FIG. | 500 | 505 | 510 | 515 | 520 | 525 | | | | | | | | | |
| 5 | | | | | | | | | | | | | | | |
| FIG. | 600 | 605 | 610 | 615 | 620 | 625 | | | | | | | | | |
| 6 | | | | | | | | | | | | | | | |
| FIG. | 700 | 705 | 710 | 715 | | | | | | | | | | | |
| 7 | | | | | | | | | | | | | | | |
| FIG. | 800 | 805 | 810 | 815 | | | | | | | | | | | |
| 8 | | | | | | | | | | | | | | | |
| FIG. | 900 | 905 | 910 | 915 | | | | | | | | | | | |
| 9 | | | | | | | | | | | | | | | |
| FIG. | 1000 | 1005 | 1010 | 1015 | | | | | | | | | | | |
| 10 | | | | | | | | | | | | | | | |
| FIG. | 1100 | 1105 | 1110 | | | 1025 | | | | | | | | | |
| 11 | | | | | | | | | | | | | | | |
| FIG. | 1200 | 1205 | 1210 | 1215 | 1220 | 1225 | | | | | | | | | |
| 12 | | | | | | | | | | | | | | | |
| FIG. | 1300 | 1305 | 1310 | | 1320 | 1325 | | | | | | | | | |
| 13 | | | | | | | | | | | | | | | |
| FIG. | 1400 | 1405 | 1410 | | 1420 | 1425 | | | | | | | | | |
| 14 | | | | | | | | | | | | | | | |
| FIG. | 1500 | 1505 | 1510 | | 1520 | | | | | 1540 | 1545 | | 1555 | | |
| 15 | | | | | | | | | | | | | | | |
| FIG. | 1600 | 1605 | 1610 | 1615 | 1620 | 1625 | | | | | | | | | |
| 16 | | | | | | | | | | | | | | | |
| FIG. | 1700 | | | | | | | | | | | | | | |
| 17 | | | | | | | | | | | | | | | |
| FIG. | 1800 | | | | | | | | | | | | | | |
| 18 | | | | | | | | | | | | | | | |
| FIG. | 1900 | 1905 | 1910 | 1915 | | | | | | | | | | | |
| 19 | | | | | | | | | | | | | | | |
| FIG. | 2000 | 2005 | 2010 | 2015 | | | | | | | | | | 2060 | |
| 20 | | | | | | | | | | | | | | | |
| FIG. | 2100 | | | | | | | | | | | | | | |
| 21 | | | | | | | | | | | | | | | |
| FIG. | 2200 | | | | | | | | | | | | | | |
| 22 | | | | | | | | | | | | | | | |
| FIG. | 2300 | 2305 | 2310 | 2315 | 2320 | 2325 | | | | | | | | | |
| 23 | | | | | | | | | | | | | | | |
| FIG. | | 2405 | 2410 | 2415 | 2420 | 2425 | | | | | | | | | |
| 24 | | | | | | | | | | | | | | | |
| FIG. | | 2505 | 2510 | 2515 | 2520 | 2525 | 2530 | 2535 | | | | | | | |
| 25 | | | | | | | | | | | | | | | |
| FIG. | | 2605 | 2610 | 2615 | 2620 | 2625 | 2630 | 2635 | | | | | | | 2665 |
| 26 | | | | | | | | | | | | | | | |
| FIG. | | 2705 | 2710 | 2715 | | | | | | | | | | | |
| 27A- | | | | | | | | | | | | | | | |
| D | | | | | | | | | | | | | | | |

### DETAILED DESCRIPTION

The present disclosure relates to a curved linkage system for a shoulder brace that provides the benefit of static stabilization offered by known braces while also advantageously permitting abduction, rotational control, stabilization during movement, and dynamic range of motion movements and exercises that are not possible while wearing known braces. Known braces generally include a waist plate that is affixed to an arm support, which immobilizes the arm of a wearer. Disclosed curved linkage systems include a series of curved link pairs that attach to and permit dynamic positional adjustment of an arm support with respect to a waist plate. The disclosed curved linkage systems permit a wearer to dynamically adopt positions including, but not limited to, a gunslinger, a neutral plane, and a statue of liberty position while performing rehabilitation exercises. Further, the curved linkage systems allow the user to have a rotational pivoting axis around their humerus while having a 90 degree (or about 90 degree) bent elbow that always points generally at the glenohumeral joint in the shoulder, giving a much more anatomic alignment. Additionally, the disclosed curved linkage system is lightweight, inexpensive to manufacture, has L3960 coding “airplane design,” convertible to right or left arm shoulder braces, and is durable.

Disclosed curved linkage systems may be made of any polymer, including injection molded nylon or glass filled nylon plastics. For example, in some embodiments, the curved linkage system may include at least one curved link pair, which may be made from a polymer comprising polyethylene, a polystyrene, a nylon, a molded nylon, a glass filled nylon plastic, a polypropylene, a polyvinylchloride, a neoprene, a cellulose, a rubber, a silicone, a tetrafluoroethylene, a polyamide, a polybutadiene, and copolymers thereof. In some embodiments, the curved linkage system may include at least one curved link pair made from a metal. For example, the curved linkage system may include at least one curved link pair made from a metal including steel, iron, aluminum, titanium, zinc, bronze, chromium, tin, tungsten, zinc, and alloys thereof. The curved link pair may also be made from a combination of a metal and a polymer.

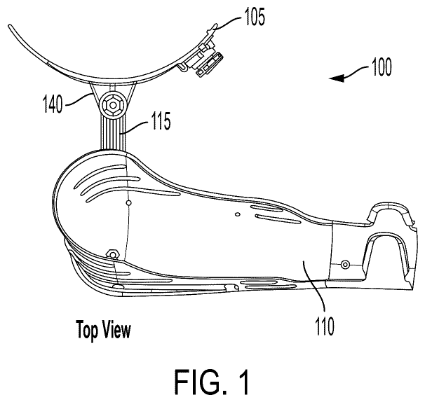

FIGS. 1-4 show various views of a shoulder brace 100, 200, 300, 400 having a disclosed curved linkage system 115, 215, 315, 415. FIG. 1 is a top view of the shoulder brace 100. The shoulder brace 100 may include a curved linkage system 115 that connects a waist plate 105 to an arm support 110. Advantageously, a rotational position of the curved linkage systems 100, with respect to the waist plate 105, may be adjusted in 15 degree increments about their connection sites. For example, the curved linkage system 100, with respect to the waist plate 105 may be adjusted at an angle of about 15 degrees, or about 30 degrees, or about 45 degrees, or about 60 degrees, or about 75 degrees, or about 90 degrees, or about 105 degrees, or about 120 degrees, or about 135 degrees, or about 150 degrees, or about 165 degrees, or about 180 degrees, or more, where about includes plus or minus 7.5 degrees. Advantageously, a rotational position of the curved linkage systems 100, with respect to the arm support 110, may be adjusted in 15 degree increments about their connection sites. For example, the curved linkage system 100, with respect to the arm support 110 may be adjusted at an angle of about 15 degrees, or about 30 degrees, or about 45 degrees, or about 60 degrees, or about 75 degrees, or about 90 degrees, or about 105 degrees, or about 120 degrees, or about 135 degrees, or about 150 degrees, or about 165 degrees, or about 180 degrees, or more, where about includes plus or minus 7.5 degrees. In some embodiments, a curved linkage system 115, 215, 315, 415 may include any number of curved link pair, where the rotational position of one curved link pair with respect to another curved link pair, may be adjusted at an angle of about 15 degrees, or about 30 degrees, or about 45 degrees, or about 60 degrees, or about 75 degrees, or about 90 degrees, or about 105 degrees, or about 120 degrees, or about 135 degrees, or about 150 degrees, or about 165 degrees, or about 180 degrees, or more, where about includes plus or minus 7.5 degrees.

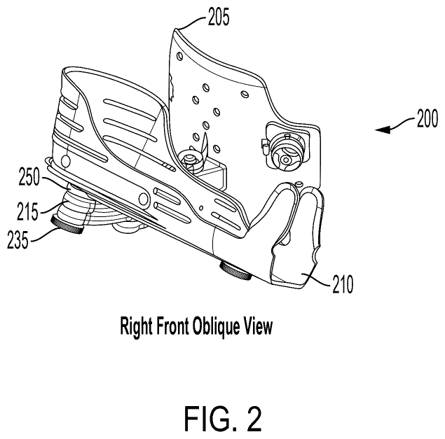

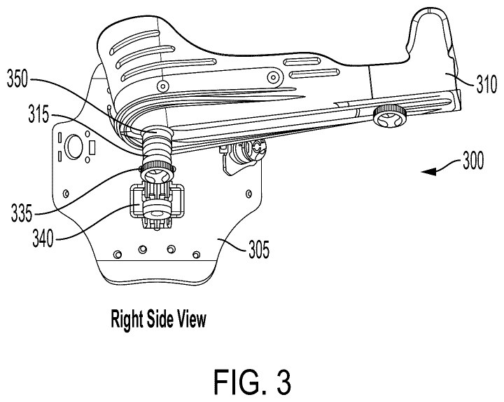

As shown in FIG. 1, a curved linkage system 115 may connect to a waist plate 105 at support lug 140 that protrudes form the waist plate 105 and has a vertical axial hole for receiving a throughbolt to connect the components. The support lug 140 may be configured to receive a throughbolt that passes through a cylindrical joint of the curved linkage system 115, thereby joining the two components. The through bolt can be fastened with a nut to secure the connection. Additionally, to provide for 15 degree increment rotational adjustment capabilities, friction washers and/or serrated disks may be included at the connection site of the support lug 140 and the curved linkage system 115. FIG. 2 is a right front oblique view of the shoulder brace 200, illustrating how the curved linkage system 215 connects to a lower portion of the arm 210 through a pivot post 250 that protrudes from a bottom surface of the arm support 210. FIG. 2 also illustrates how the curved linkage system 215 connects to waist plate 205. The curved linkage system 215 may connect to a bottom portion of the arm support 210 through a knob 235 having a throughbolt that inserts and threadably screws into a pivot post 250 protruding from a bottom surface of the arm support 210. To provide for 15 degree increment rotational adjustment capabilities at the connection site between the arm support 210 and the curved linkage system 215, friction washers and/or serrated disks may be incorporated. By adjusting how tight the knob 235 is secured to the pivot post 450, a user may adjust the level of incremental rotational adjustment of the arm support 210 with respect to the curved linkage system 215. FIG. 3 illustrates a right side view of the curved linkage system 315 of FIG. 1 and FIG. 2. As shown in FIG. 3, the shoulder brace 300 may include knob 335 having a throughbolt may secure the distal joint of the second curved link pair to the pivot post 350. FIG. 3 also shows how the curved linkage system 315 may connect the arm support 310 to the waist plate 305. As shown in FIG. 3, the shoulder brace 300 may include a support lug 340 protruding from the waist plate 305 and is configured to connect to a first curved link through a proximal cylindrical joint.

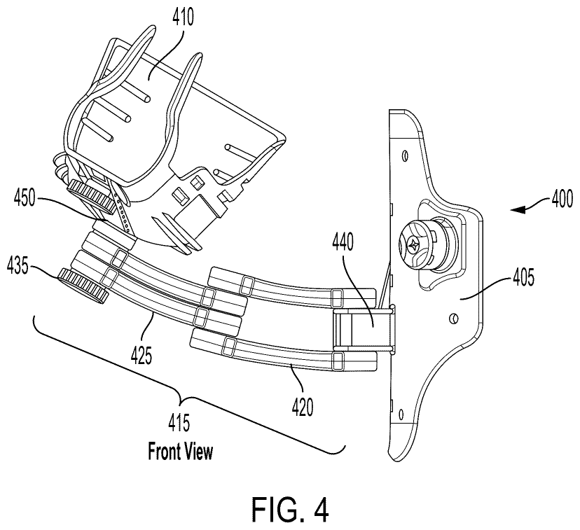

FIG. 4 illustrates a front view of a shoulder brace 400, showing an extended curved linkage system 415. As shown in FIG. 4, the curved linkage system 415 may include a first curved link pair 420 and a second curved link pair 425 that each have an upper curved link and a lower curved link. Each of the upper curved link and lower curved link pair may contain two cylindrical joints located at each end. For example, each curved link pair may contain a proximal cylindrical joint and a distal cylindrical join (with respect to a connection to a waist plate 405) connected by an elongated body. The first curved link pair 420 may connect to a support lug 440 protruding from the waist plate 405. The connection between the first curved link pair 420 and the support lug 440 may be made by passing a throughbolt through the proximal cylindrical joints of the first curved link pair 420 and the support lug 440 and then securing the connection with a nut or any other known fastener. The distal cylindrical joints of the first curved link pair 420 may connect to the proximal cylindrical joints of the second curved link pair 425. Throughbolts and nuts may secure the connection between the first curved link pair 420 and the second curved link pair 425. The curved links of the first curved link pair 420 may be positioned in parallel so the proximal cylindrical joints, the elongated bodies, and the distal cylindrical joints are each aligned with each other, as shown in FIG. 4. Similarly, the curved links of the second curved link pair 425 may be positioned in parallel so that the proximal cylindrical joints, the elongated bodies, and the distal cylindrical joints are each aligned with each other.

In some embodiments, proximal cylindrical joints of a second curved link pair fit inside of distal cylindrical joints of a first curved link pair when connected. This staggered configuration advantageously permits full rotation about each connection site without the curved link pairs pushing into each other. Additionally, a pivoting position where the two curved link pairs connect may include a frictional device with a fastener to permit approximate 15 degree incremental adjustments. As shown in FIG. 4, distal joints of the second curved link pair 425 may connect to a pivot post 450 protruding from a bottom surface of an arm support 410. Knob 435 having a throughbolt may secure the distal joint of the second curved link pair 425 to the pivot post 450.

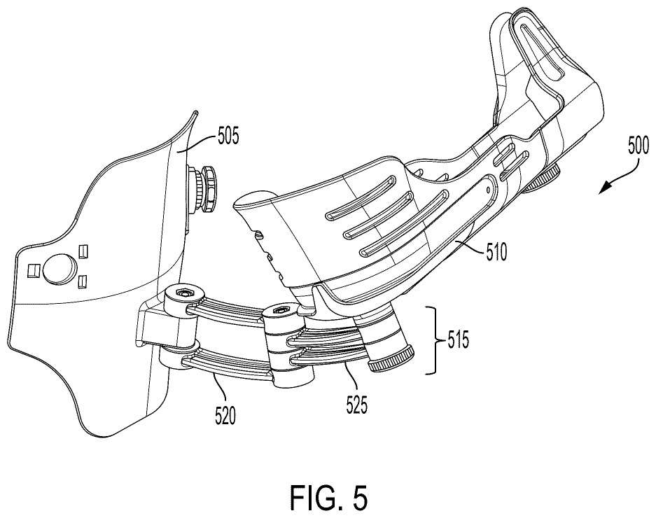

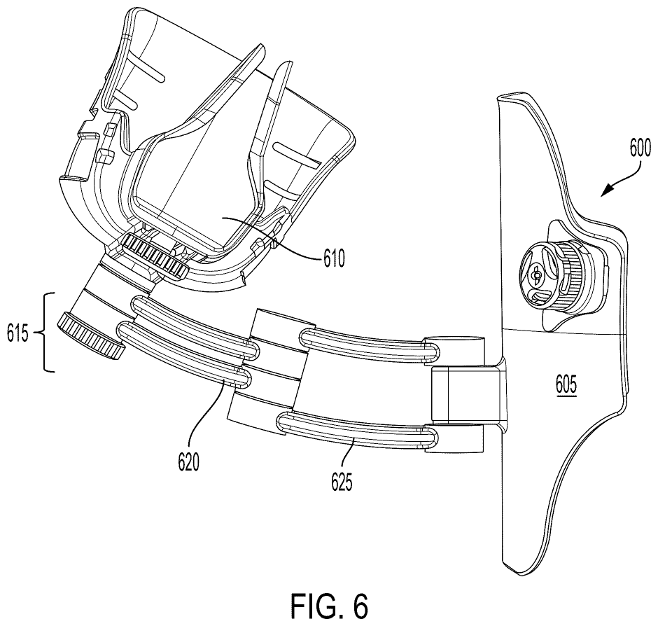

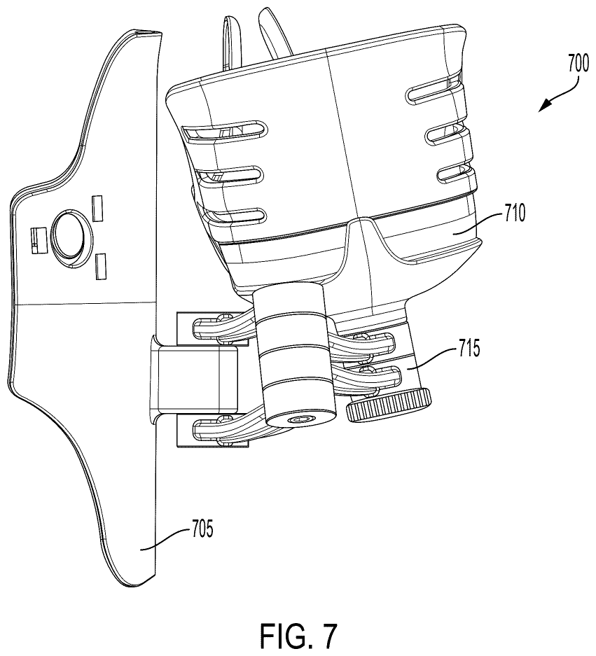

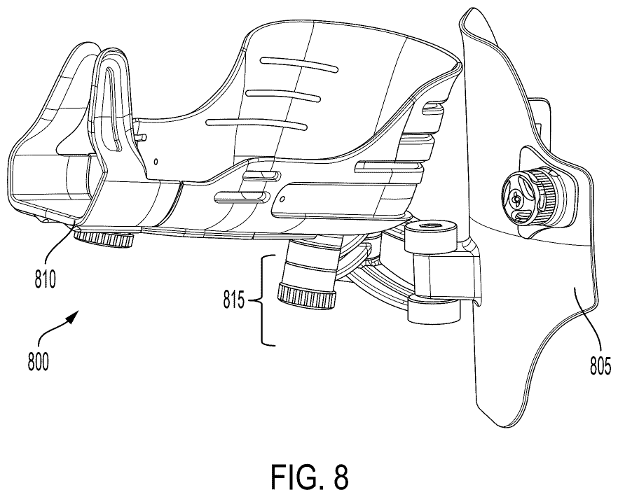

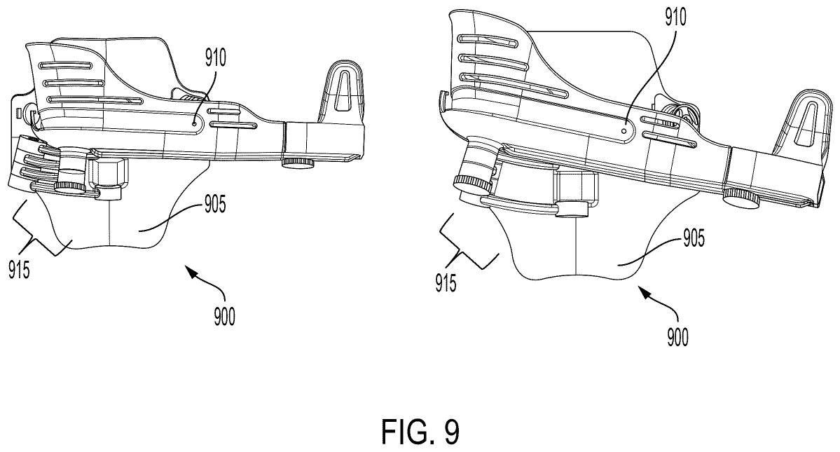

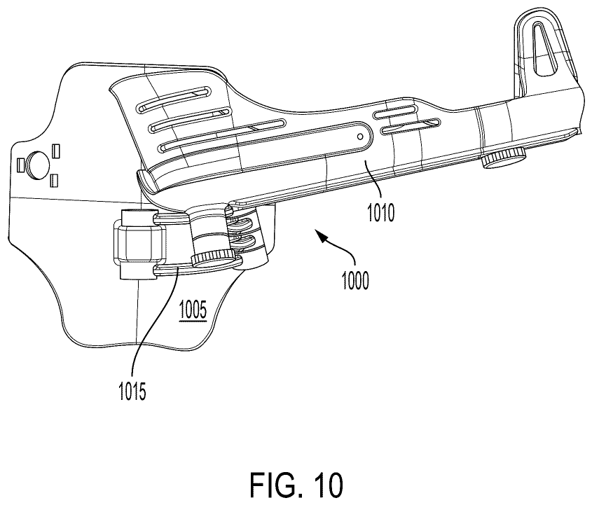

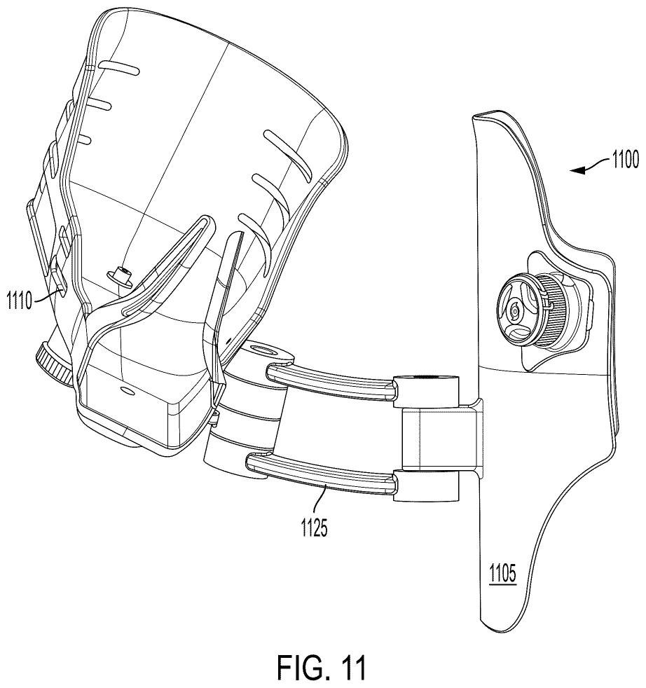

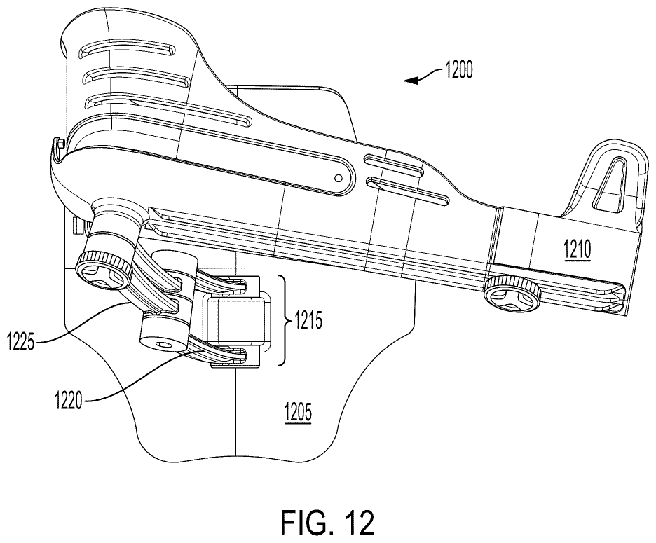

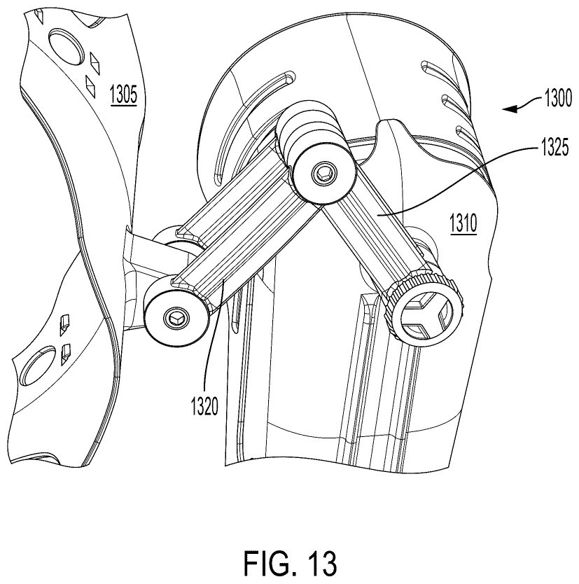

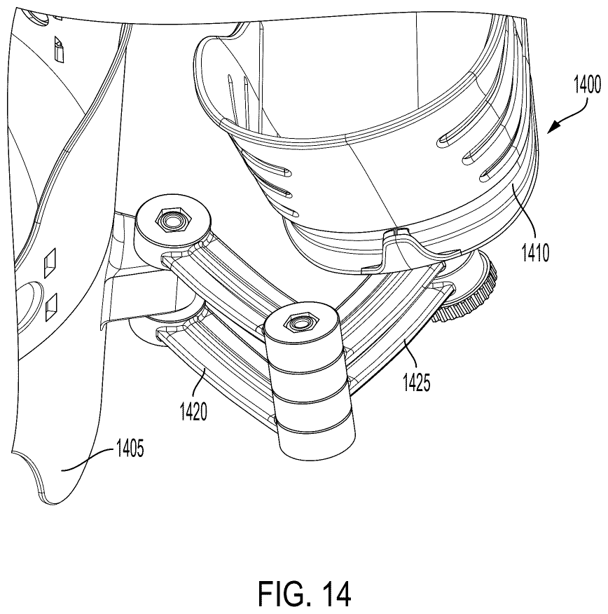

A disclosed curved linkage system may advantageously form an adjustment mechanism between a waist plate and an arm support to allow movement of a user's arm in various positions. Each curved link pair may provide for an approximate 12 inch radius of movement, as shown in FIGS. 5-16. The 12 inch radius is an average distance from a bottom of an elbow to a shoulder joint of a user. In some embodiments, each curved link pair may provide for an about 9 inch radius of movement, or an about 10 inch radius of movement, or an about 11 inch radius of movement, or an about 12 inch radius of movement, or an about 13 inch radius of movement, or an about 14 inch radius of movement, or an about 15 inch radius of movement, or an about 16 inch radius of movement, where about includes plus or minus 0.5 inches. FIG. 5 illustrates the shoulder brace 500 having a curved linkage system 515 that is fully extended out from the waist plate 505 so that the first curved link pair 520 and the second curved link pair 525 are in a straight line and extending perpendicular to waist plate 405. However, even though the arm support 510 is shown in FIG. 5 to be aligned perpendicular to the curved linkage system 515, the arm support 510 may be rotated freely about its connection to the curved linkage system 515 as needed. FIG. 6 illustrates a front view of the fully extended out curved linkage system 615. In FIG. 6, the first curved link pair 620 and the second curved link pair 625 is oriented substantially in a straight line and arranged perpendicular to the arm support 610 and the waist plate 605 of the shoulder brace 600. As shown in FIG. 7, the curved linkage system 715 of the shoulder brace 700 may be collapsed onto itself through rotation about each connection site so that the arm support 710 is nearly touching the waist plate 705. FIG. 8 illustrates a front side view of the shoulder brace 800 where the curved linkage system 815 has been folded onto itself so that the arm support 810 is nearly touching the waist plate 805. FIG. 9 illustrates two side views with the first view having the same collapsed configuration shown in FIGS. 7-8 and the second view having the curved linkage system 915 slightly opened up. FIG. 9 illustrates the shoulder brace 900 where the curved linkage system 915 has been folded onto itself so that the arm support 910 is nearly touching the waist plate 905. FIG. 10 is a side view of the second configuration shown in FIG. 9, where the curved linkage system 1015 of the shoulder brace 1000 has been slightly opened up so that the arm support 1010 is not as close to the waist plate 1005 as in the more collapsed configurations shown in FIGS. 7-8. FIG. 11 shows a front view of the shoulder brace 1100 configuration shown in FIG. 10. FIG. 11 illustrates shoulder brace 1100 having the arm support 1110 connected to the waist plate 1105, where the second curved link pair 1025 is visible. FIG. 12 shows a side view of the shoulder brace 1200 where the curved linkage system 1215 is opened up so that the first curved link pair 1220 and the second curved link pair 1225 are in a straight line while extending perpendicular to waist plate 1205. The first curved link pair 1220 and the second curved link pair 1225 are oriented perpendicular with respect to the waist plate 1205 and the arm support 1210. FIG. 13 illustrates a bottom view of the shoulder brace 1300 where the first curved link pair 1320 is oriented at an about 90 degree angle with respect to the waist plate 1305. The second curved link pair 1325 is oriented at an about 90 degree angle with respect to the first curved link pair 1320 that is connected to the waist plate 1310. FIG. 14 is a top view of a similar configuration of the shoulder brace 1400 as shown in FIG. 13, where the first curved link pair 1420 is oriented at an about 90 degree angle with respect to the waist plate 1405. The second curved link pair 1425 is oriented at an about 90 degree angle with respect to the first curved link pair 1420 that is connected to the waist plate 1410.

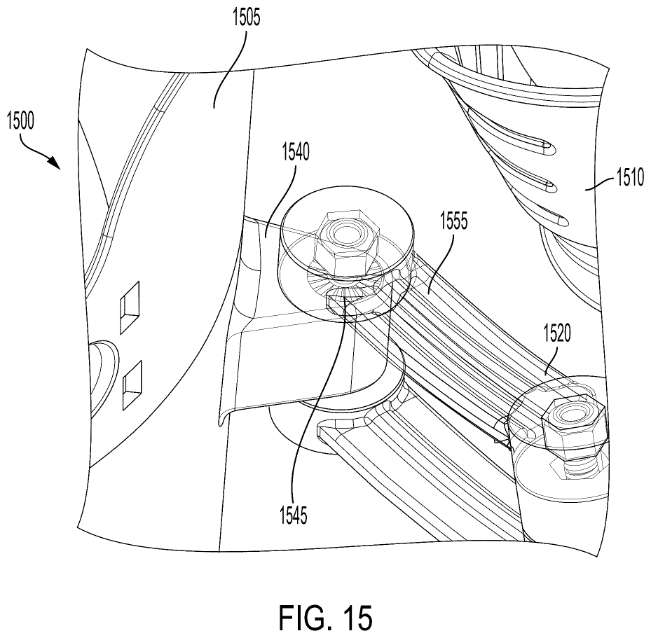

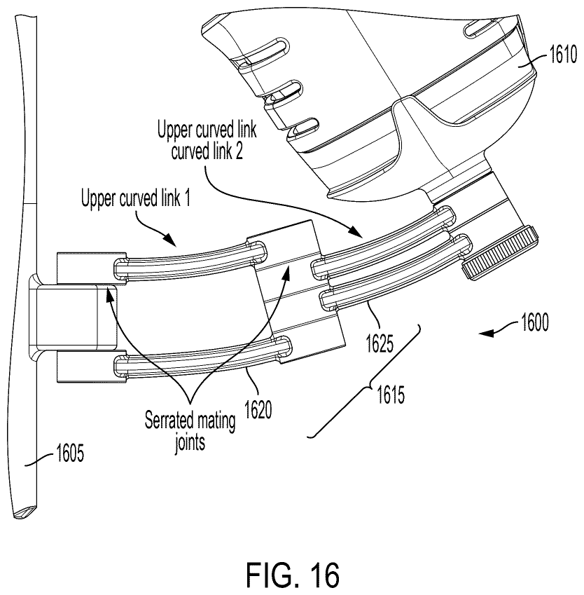

FIG. 15 illustrates an x-ray view of how an upper curved link 1555 of a first curved link pair 1520 may connect to a support lug 1540 protruding from a waist plate 1505 of a shoulder brace 1500. As shown in FIG. 15, the shoulder brace 1500 may include an arm support 1510. As shown in FIG. 15, an upper curved link 1555 may connect to the support lug 1540 while having a serrated disk 1545 present. In some embodiments, serrated disks 1545 may only be included in the joints with the upper curved link 1555, such as the upper connection joints. In some embodiments, serrated disks 1545 may be excluded from lower connection joints and/or middle connection joints. FIG. 16 illustrates a side view of an shoulder brace 1600 having a fully extended curved linkage system 1615. As shown in FIG. 16, in some embodiments, serrated disks are only found in the top connection point. As shown in FIG. 16, shoulder brace 1600 may include a waist plate 1605 connected to an arm support 1610 through a curved linkage system 1615 having a first curve link pair 1620 and a second curved link pair 1625 that are placed in a linear orientation.

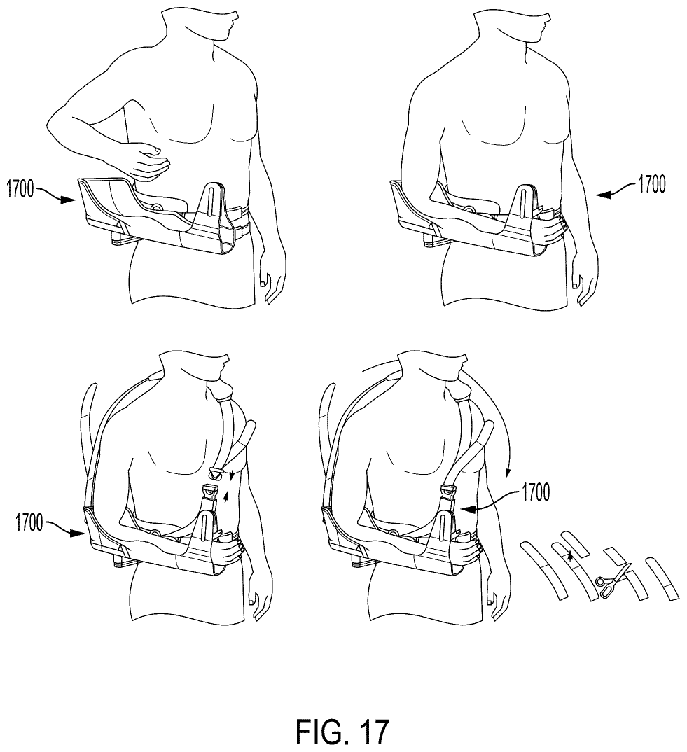

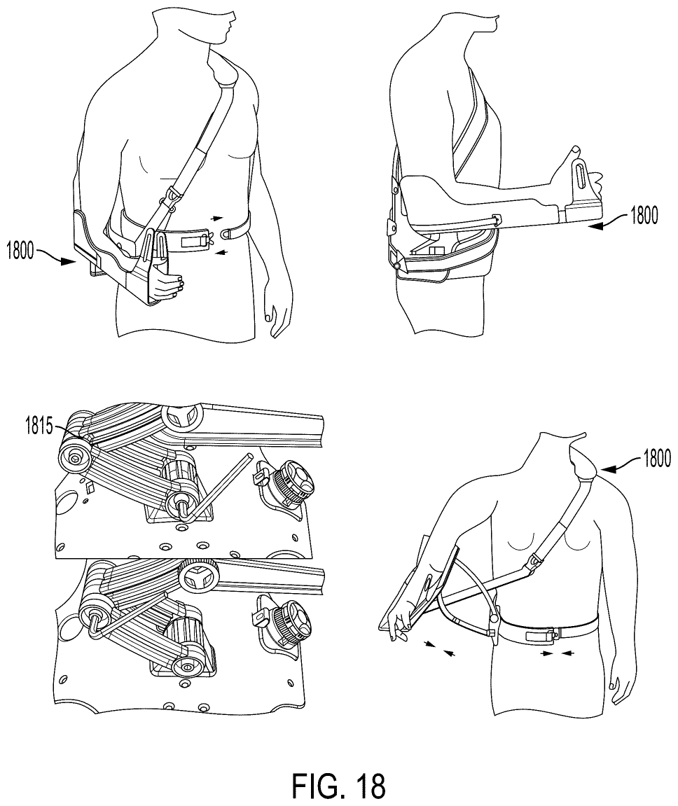

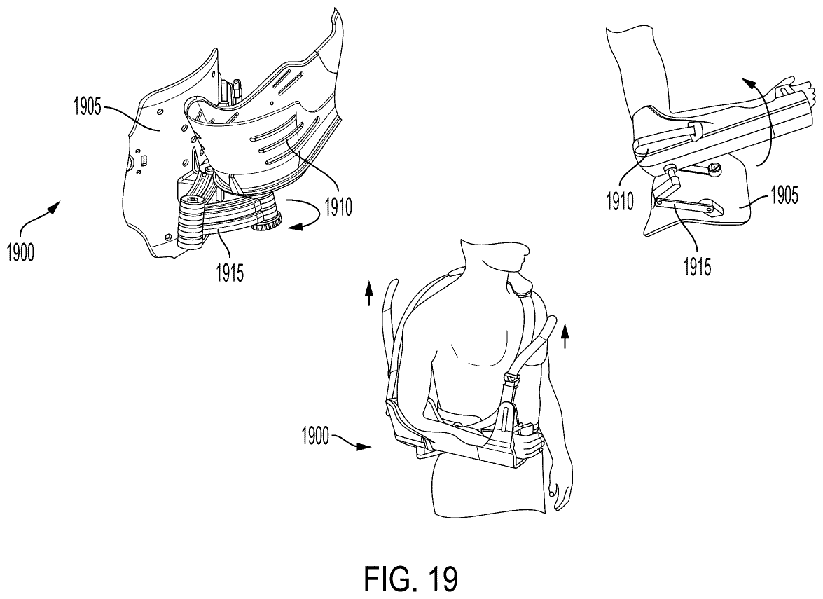

FIGS. 17-19 illustrate various steps of using a shoulder brace 1700-1900 having a disclosed curved linkage system. Installation steps may include positioning a hip pad onto a hip with a bottom of elbow bucket at elbow level, and slipping an arm into an arm support to ensure the elbow is seated into the elbow bucket area. The next step includes securing the shoulder strap by passing the shoulder strap over the shoulder and inserting the buckle at the wrist support strap. After this, adjusting the shoulder strap of the shoulder brace 1700 shown in FIG. 17 at a front and back by loosening hook closures and pulling strap ends to a comfortable tension. As shown in FIG. 18, the waist strap can be adjusted by fastening the waist strap around the waist. The arm length may be adjusted by loosening the handgrip release knob in a counterclockwise direction underneath the forearm support until the handgrip can slide easily. Then, the handgrip can be lengthened or shortened until the handgrip sits comfortably in the palm. The handgrip knob may be tightened by turning it in a clockwise direction and loosened by turning it in a counter-clockwise direction. As shown in FIG. 18, a shoulder brace 1800 may be adjusted by loosening the socket head cap screws at a bottom of the curved links at a bottom of a support lug protruding from a waist plate. Similar adjustments may be made to the connection between the curved link pairs by using a hex key wrench. After positioning of the forearm support to a desired position taking advantage of the curved link pairs having an adjustment index being in about 15 degree angles, the screws may be re-tightened to secure the position of the components relative to each other. As shown in FIG. 19, rotation of the arm support 1910 of the shoulder brace 1900 may be accomplished by loosening a knob located at a bottom of the curved linkage system 1915, rotating the arm support 1910 to a desired position, and then tightening the knob back. As shown in FIG. 19, final adjustments may be made to the shoulder straps.

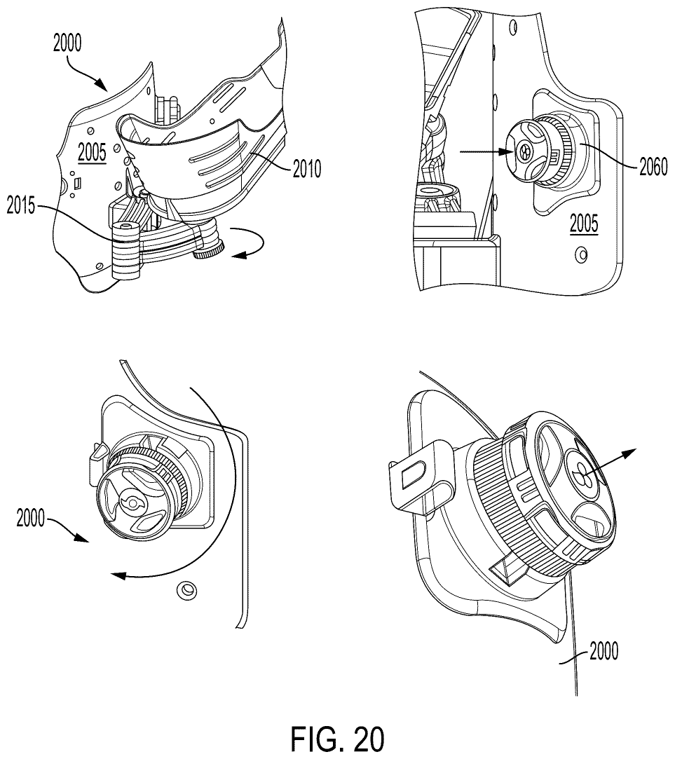

FIG. 20 illustrates how dynamic tensioning of shoulder brace 2000 may be accomplished. As shown in FIG. 20, the shoulder brace 2000 may include a dynamic tension knob 2060 located on waist plate 2005, which may be may be rotated to increase or decrease tension throughout the shoulder brace 2000. FIG. 20 also shows the orientation of the curved linkage system 2015 and the arm support 2010 with respect to the waist plate 2006 and dynamic tension knob 2060.

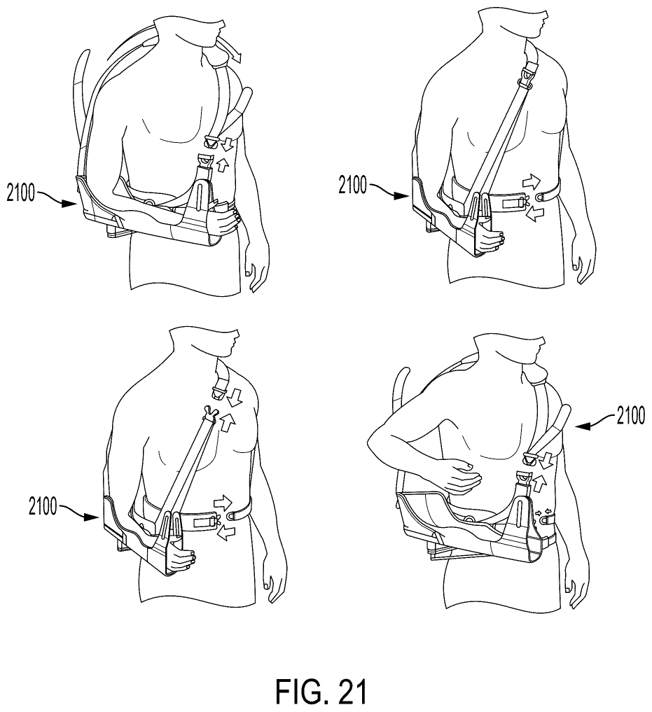

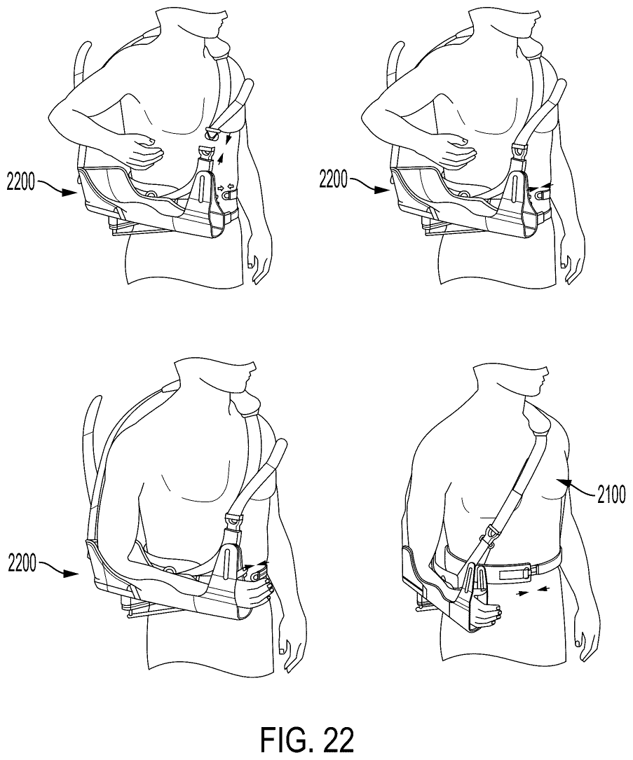

FIG. 21 illustrates how to remove a disclosed shoulder brace 2100. Steps for removing the shoulder brace 2100 may include loosening the shoulder strap so that enough length is obtained so that a strap may be brought overhead or completely released from the buckle. The waistband buckle may then be released, followed by sliding of an arm out of the shoulder brace 2100, and then fully removing the shoulder brace 2100. FIG. 22 shows steps of how to reapply the removed shoulder brace 2200 by bringing the loosened but attached shoulder strap overhead, and then tightening loose shoulder straps. Then, by bringing the arm into the arm support and into the elbow socket, and then by attaching the waistband to buck and tighten as needed, the shoulder brace 2200 is reapplied.

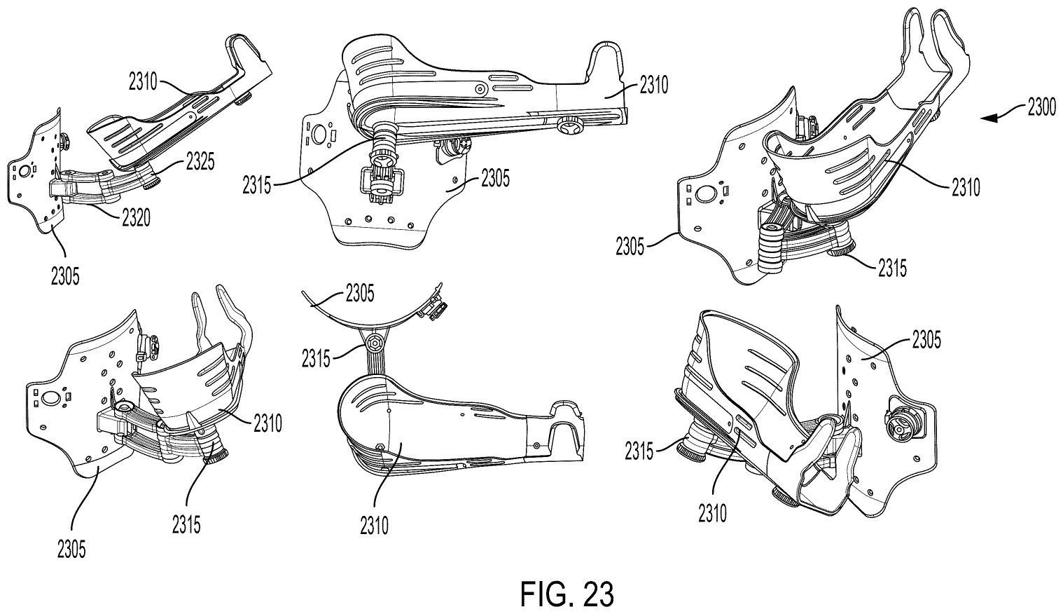

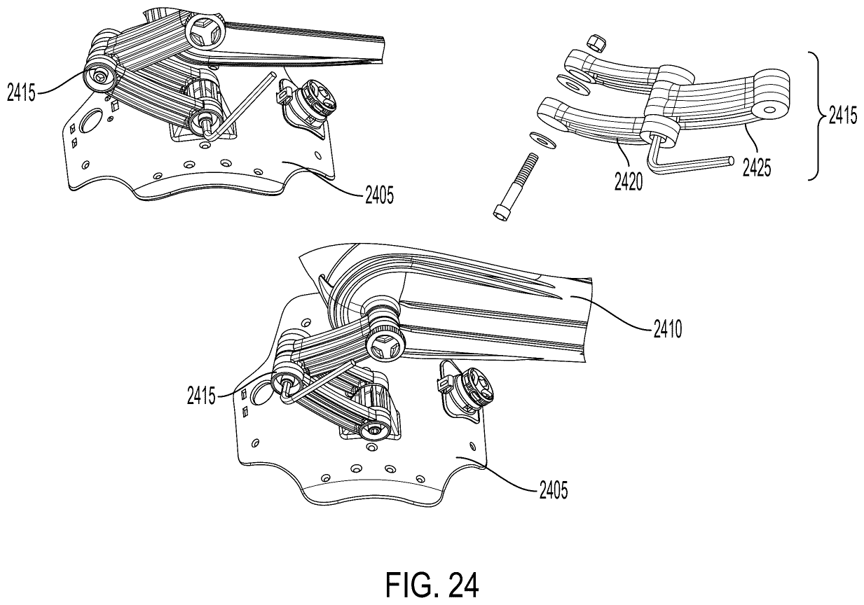

FIGS. 23-24 illustrate other adjustment points for disclosed shoulder braces 2300-2400. For example, FIG. 23 depicts various views of a fully collapsed and fully extended curved linkage system 2315 in various orientations, showing different angles in which the waist plate 2305, arm support 2310, first curved link pair 2320, and second curved link pair 2325 can be placed with respect to each other and other components of a disclosed shoulder brace 2300. FIG. 24 illustrates adjustment points of the curved linkage system 2415 along with a slightly exploded view of the bolt, washer, and nut connection components used when adjusting the curved linkage system 2415. As shown in FIG. 24, the first curved link pair 2420 and the second curved link pair 2425 can be assembled to form the curved linkage system 2415, attached to a waist plate 2405 and/or an arm support 2410, and adjusted using known components including bolts, washers, and nuts.

Method for Extending from Two Curved Link Pairs to Four Curved Link Pairs

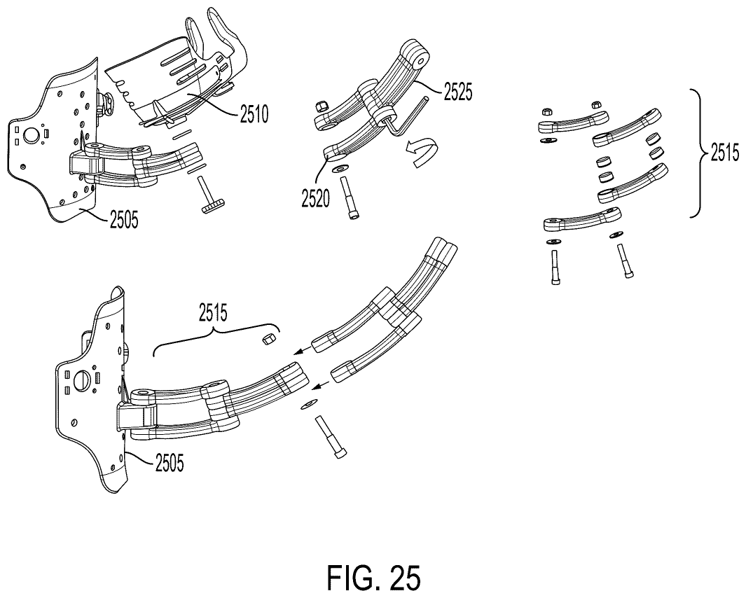

The present disclosure relates to methods for extending and positioning a curved linkage system, as shown in FIGS. 25-27. As shown in FIG. 25, curved linkage system 2515 may connect a waist plate 2505 to an arm support 2510. The disclosed method may exclude increasing the number of curved linkage pairs from 2 (FIG. 25) to 3 or 4 (FIGS. 25-27). For example, the curved linkage system 2515 including a first curved link pair 2520 including an upper curved link and a lower curved link that each include a proximal cylindrical joint and a distal cylindrical joint connected by an elongated bode. The two curved links may be positioned in parallel so the proximal cylindrical joints, the elongated bodies, and the distal cylindrical joints may be each aligned with each other. The first curved linked pair 2520 may be configured to connect to a support lug protruding from the waist plate through the proximal cylindrical joints. The distal cylindrical joints of the first curved link pair 2520 may be configured to connect to proximal cylindrical joints of a second curved link pair. The curved linkage system 2515 may include a second curved link pair 2525 including an upper curved link and a lower curved link that each include a proximal cylindrical joint and a distal cylindrical joint connected by an elongated body. As shown in FIG. 25, the two curved links may be positioned in parallel so that the proximal cylindrical joints, the elongated bodies, and the distal cylindrical joints may be each aligned with each other. The proximal cylindrical joints of the second curved link pair 2525 may fit inside the distal cylindrical joints of the first curved link pair 2520 when connected. The curved linkage system 2515 may include a third curved link pair 2530 including an upper curved link and a lower curved link that each include a proximal cylindrical joint and a distal cylindrical joint connected by an elongated body. The two curved links may be positioned in parallel so that the proximal cylindrical joints, the elongated bodies, and the distal cylindrical joints may be each aligned with each other. As shown in FIG. 25, the proximal cylindrical joints of the second curved link pair 2525 may fit inside the distal cylindrical joints of the second curved link pair 2525 when connected. The curved linkage system 2515 may include a fourth curved link pair 2535 including an upper curved link and a lower curved link that each include a proximal cylindrical joint and a distal cylindrical joint connected by an elongated body. As shown in FIG. 25, the two curved links may be positioned in parallel so that the proximal cylindrical joints, the elongated bodies, and the distal cylindrical joints may be each aligned with each other. The proximal cylindrical joints of the second curved link pair 2525 may fit inside the distal cylindrical joints of the third curved link pair 2530 when connected. Distal joints of the fourth curved link pair 2535 may be configured to connect to a pivot post protruding from a bottom surface of the arm support 2510.

As shown in FIG. 25, a knob having a throughbolt may pass through washers and a cylindrical joint of a curved link pair to couple the curved link pair to a bottom of an elbow forearm rest. A first step to extending a curved linkage system 2515 may include disengaging the arm support 2510 from the cylindrical joint of one of the curved link pairs of the curved linkage system 2515 by removing the knob from the cylindrical joint. As shown in FIG. 25, a plastic washer may separate a head of the knob from a bottom surface of the cylindrical joint and an elastomeric washer may separate a top surface of the cylindrical joint from a pivot post located on a bottom surface of the elbow forearm rest. During this step, the arm support 2510 may remain connected to a tension cable on a cable reel at a front side of the waist plate. To extend the number of curved link pairs, two more curved link pairs (third curved link pair 2530, fourth curved link pair 2535) may be fitted over the cylindrical joint and then coupled to one of the first curved link pair 2520 and the second curved link pair 2525 by inserting a bolt through the connecting cylindrical joints and then securing the bolt with a nut, thereby providing four total curved link pairs extending from the waist plate.

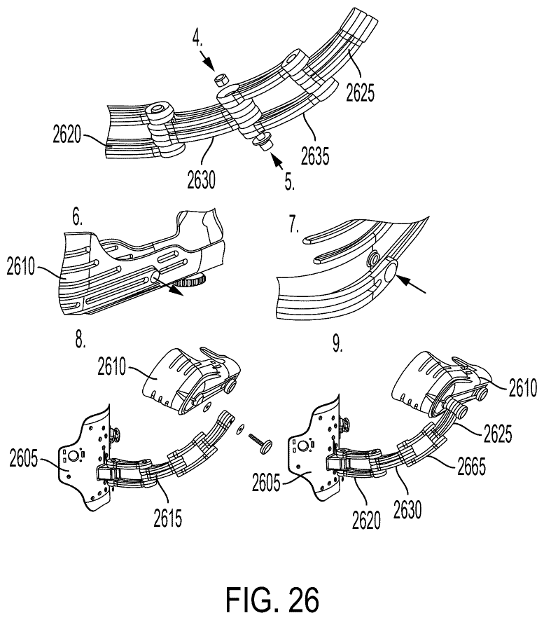

A wrench (e.g., hex-head wrench) may be used to threadably tighten and secure the bolt to a nut to link multiple curved link pairs through their cylindrical joints. In some embodiments, the nut may be fitted into a hole recessing into a surface of one of the cylindrical joints. As shown in FIG. 26, by aligning the bolt through holes of each cylindrical joint and the nut, each of the components may be coupled. In some embodiments, washers (e.g., metal, plastic, elastomeric) may be used in between components. The method may include removing a tension band snap from a forward male snap on a side of the arm cradle, as shown in FIG. 26. The method may include pressing a tension band snap into a rearward male snap on the arm support 2610 to permit greater abduction. The method may include replacing an arm support 2610 on the fourth curved link pair 2635 using the knob, plastic washer, and elastomeric washer removed in earlier steps. As shown in FIG. 26, the arm support 2610 connects to the fourth curved link pair 2635, which connects to the third curved link pair 2630, which connects to the second curved link pair 2625, which connects to the first curved link pair 2620 (curved link pairs combined to make curved linkage system 2615), which may connect to the waist plate 2605. In some embodiments, an avoidance of twisting of a cable tension (not shown) between the tension adjuster knob and back of the forearm elbow cradle. The method may include adjusting a shoulder abduction angle and position of the elbow forearm rest with respect to the waist plate.

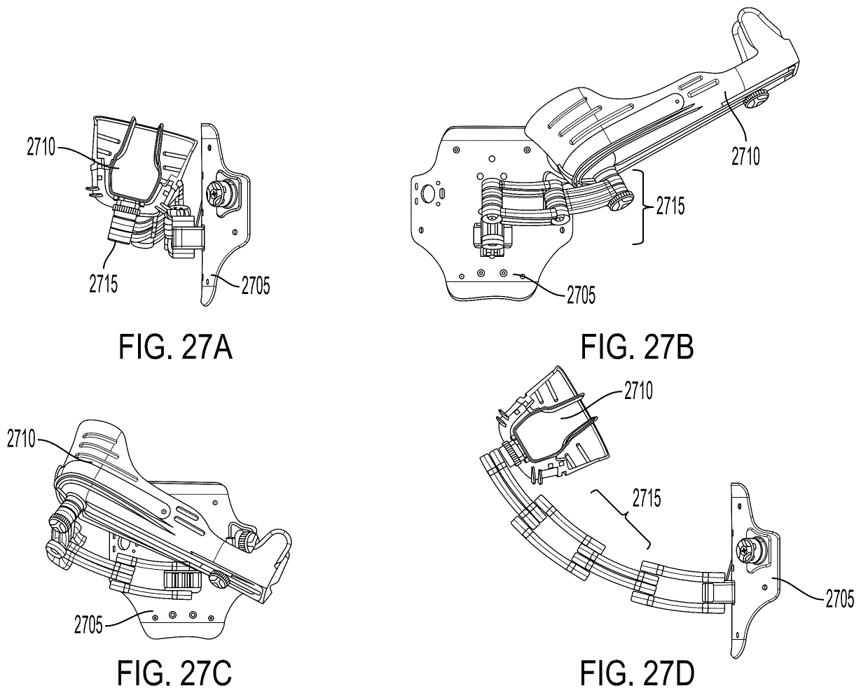

According to some embodiments, as shown in FIG. 27, the present disclosure relates to methods for adjusting a position of an arm support 2710 with respect to a waist plate 2705 by adjusting the curved linkage system 2715. As shown in FIG. 27, the arm support 2710 may be repositioned in 15-degree increments about the curved link pairs. FIG. 27A illustrates a minimum abduction conformation whereas FIG. 27D illustrates a maximum abduction conformation where the curved link pairs are fully extended laterally and in a neutral position. FIG. 27B and FIG. 27C show various conformations in between those shown in FIG. 27A and FIG. 27D. FIG. 27B illustrates a side view of the disclosed system with curved link pairs positioned anteriorly with respect to the arm support 2710. The arm support 2710 may be rotated so that the curved link pairs are positioned posteriorly with respect to the arm support 2710, as shown in FIG. 27C.

As will be understood by those skilled in the art who have the benefit of the instant disclosure, other equivalent or alternative compositions, devices, and disclosed curved linkage systems can be envisioned without departing from the description contained in this application. Accordingly, the manner of carrying out the disclosure as shown and described is to be construed as illustrative only.

Persons skilled in the art can make various changes in the shape, size, number, and/or arrangement of parts without departing from the scope of the instant disclosure. For example, the position and number of curved linkages can be varied. In some embodiments, curved linkages can be interchangeable. In addition, the size of a device and/or system can be scaled up or down to suit the needs and/or desires of a practitioner. Each disclosed process, system, method, and method step can be performed in association with any other disclosed method or method step and in any order according to some embodiments. Where the verb “may” appears, it is intended to convey an optional and/or permissive condition, but its use is not intended to suggest any lack of operability unless otherwise indicated. Where open terms such as “having” or “comprising” are used, one of ordinary skill in the art having the benefit of the instant disclosure will appreciate that the disclosed features or steps optionally can be combined with additional features or steps. Such option may not be exercised and, indeed, in some embodiments, disclosed systems, compositions, apparatuses, and/or methods can exclude any other features or steps beyond those disclosed in this application. Elements, compositions, devices, systems, methods, and method steps not recited can be included or excluded as desired or required. Persons skilled in the art can make various changes in methods of preparing and using a composition, device, and/or system of the disclosure.

Also, where ranges have been provided, the disclosed endpoints can be treated as exact and/or approximations as desired or demanded by the particular embodiment. Where the endpoints are approximate, the degree of flexibility can vary in proportion to the order of magnitude of the range. For example, on one hand, a range endpoint of about 50 in the context of a range of about 5 to about 50 can include 50.5, but not 52.5 or 55 and, on the other hand, a range endpoint of about 50 in the context of a range of about 0.5 to about 50 can include 55, but not 60 or 75. In addition, it can be desirable, in some embodiments, to mix and match range endpoints. Also, in some embodiments, each figure disclosed (e.g., in one or more of the examples, tables, and/or drawings) can form the basis of a range (e.g., depicted value+/−about 10%, depicted value+/−about 50%, depicted value+/−about 100%) and/or a range endpoint. With respect to the former, a value of 50 depicted in an example, table, and/or drawing can form the basis of a range of, for example, about 45 to about 55, about 25 to about 100, and/or about 0 to about 100. Disclosed percentages are volume percentages except where indicated otherwise.

All or a portion of a disclosed curved linkage systems can be configured and arranged to be disposable, serviceable, interchangeable, and/or replaceable. These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

1. A curved linkage system for connecting an arm support to a waist plate of a shoulder brace, the curved linkage system comprising:
(a) a first curved link pair comprising an upper curved link and a lower curved link that each comprise a proximal cylindrical joint and a distal cylindrical joint connected by an elongated body,
wherein:
(i) the two curved links are positioned in parallel so the proximal cylindrical joints, the elongated bodies, and the distal cylindrical joints are each aligned with each other,
(ii) the first curved link is configured to connect to a support lug protruding from the waist plate through the proximal cylindrical joints, and
(iii) the distal cylindrical joints of the first curved link pair are configured to connect to proximal cylindrical joints of a second curved link pair; and
(b) a second curved link pair comprising an upper curved link and a lower curved link that each comprise a proximal cylindrical joint and a distal cylindrical joint connected by an elongated body,
wherein:
(i) the two curved links are positioned in parallel so that the proximal cylindrical joints, the elongated bodies, and the distal cylindrical joints are each aligned with each other,
(ii) the proximal cylindrical joints of the second curved link pair fit inside the distal cylindrical joints of the first curved link pair when connected, and
(iii) distal joints of the second curved link are configured to connect to a pivot post protruding from a bottom surface of the arm support.

2. The curved linkage system according to claim 1, further comprising a knob having a throughbolt that inserts and threadably screws into the pivot post, the knob configured to adjust a level of incremental rotational adjustment of the arm support with respect to the curved linkage.

3. The curved linkage system according to claim 1, wherein the curved linkage system further comprises:
a throughbolt that passes through part of the first curved link and a vertical axial hole of the support lug; and
a nut fastened to an end of the throughbolt to secure the first curved link to the support lug.

4. The curved linkage system according to claim 1, further comprising at least one of a serrated disk and a friction washer fitted in between the support lug and the first curved link.

5. The curved linkage system according to claim 1, further comprising a tension knob having a throughbolt configured to secure the distal joint of the second curved link pair to the pivot post.

6. The curved linkage system according to claim 1, wherein each of the first curved link pair and the second curved link pair provides for an about 12 inch radius of movement.

7. The curved linkage system according to claim 1, wherein the first curved link pair is made from a metal comprising steel, iron, aluminum, titanium, zinc, bronze, chromium, tin, tungsten, zinc, and alloys thereof.

8. The curved linkage system according to claim 1, wherein the second curved link pair is made from a metal comprising steel, iron, aluminum, titanium, zinc, bronze, chromium, tin, tungsten, zinc, and alloys thereof.

9. The curved linkage system according to claim 1, wherein the first curved link pair is made from a polymer comprising polyethylene, polystyrene, nylon, polypropylene, polyvinylchloride, neoprene, cellulose, rubber, silicone, tetrafluoroethylene, polyamide, polybutadiene, and copolymers thereof.

10. The curved linkage system according to claim 1, wherein the second curved link pair is made from a polymer comprising polyethylene, polystyrene, nylon, polypropylene, polyvinylchloride, neoprene, cellulose, rubber, silicone, tetrafluoroethylene, polyamide, polybutadiene, and copolymers thereof.

11. A curved linkage system for connecting an arm support to a waist plate of a shoulder brace, the curved linkage system comprising:
(a) a first curved link pair comprising an upper curved link and a lower curved link that each comprise a proximal cylindrical joint and a distal cylindrical joint connected by an elongated body,
wherein:
(i) the two curved links are positioned in parallel so the proximal cylindrical joints, the elongated bodies, and the distal cylindrical joints are each aligned with each other,
(ii) the first curved link is configured to connect to a support lug protruding from the waist plate through the proximal cylindrical joints, and
(iii) the distal cylindrical joints of the first curved link pair are configured to connect to proximal cylindrical joints of a second curved link pair; and
(b) a second curved link pair comprising an upper curved link and a lower curved link that each comprise a proximal cylindrical joint and a distal cylindrical joint connected by an elongated body,
wherein:
(i) the two curved links are positioned in parallel so that the proximal cylindrical joints, the elongated bodies, and the distal cylindrical joints are each aligned with each other,
(ii) the proximal cylindrical joints of the second curved link pair fit inside the distal cylindrical joints of the first curved link pair when connected, and
(c) a third curved link pair comprising an upper curved link and a lower curved link that each comprise a proximal cylindrical joint and a distal cylindrical joint connected by an elongated body,
wherein:
(i) the two curved links are positioned in parallel so that the proximal cylindrical joints, the elongated bodies, and the distal cylindrical joints are each aligned with each other,
(ii) the proximal cylindrical joints of the second curved link pair fit inside the distal cylindrical joints of the second curved link pair when connected, and
(d) a fourth curved link pair comprising an upper curved link and a lower curved link that each comprise a proximal cylindrical joint and a distal cylindrical joint connected by an elongated body,
wherein:
(i) the two curved links are positioned in parallel so that the proximal cylindrical joints, the elongated bodies, and the distal cylindrical joints are each aligned with each other,
(ii) the proximal cylindrical joints of the second curved link pair fit inside the distal cylindrical joints of the third curved link pair when connected, and
(iii) distal joints of the fourth curved link are configured to connect to a pivot post protruding from a bottom surface of the arm support.

12. The curved linkage system according to claim 11, further comprising a knob having a throughbolt that inserts and threadably screws into the pivot post, the knob configured to adjust a level of incremental rotational adjustment of the arm support with respect to the curved linkage.

13. The curved linkage system according to claim 11, further comprising:
a throughbolt that passes through part of the first curved link and a vertical axial hole of the support lug; and
a nut fastened to an end of the throughbolt to secure the first curved link to the support lug.

14. The curved linkage system according to claim 1, further comprising at least one of a serrated disk and a friction washer fitted in between the support lug and the first curved link.

15. The curved linkage system according to claim 1, further comprising a tension knob having a throughbolt configured to secure the distal joint of the second curved link pair to the pivot post.

16. The curved linkage system according to claim 1, wherein each of the first curved link pair, the second curved link pair, the third curved link pair, and the fourth curved link pair provides for an about 12 inch radius of movement.

17. The curved linkage system according to claim 1, wherein at least one of the first curved link pair and the second curved link pair is made from a metal comprising steel, iron, aluminum, titanium, zinc, bronze, chromium, tin, tungsten, zing, and alloys thereof.

18. The curved linkage system according to claim 1, wherein at least one of the third curved link pair and the fourth curved link pair is made from a metal comprising steel, iron, aluminum, titanium, zinc, bronze, chromium, tin, tungsten, zinc, and alloys thereof.

19. The curved linkage system according to claim 1, wherein at least one of the first curved link pair and the second curved link pair is made from a polymer comprising polyethylene, polystyrene, nylon, polypropylene, polyvinylchloride, neoprene, cellulose, rubber, silicone, tetrafluoroethylene, polyamide, polybutadiene, and copolymers thereof.

20. The curved linkage system according to claim 1, wherein at least one of the third curved link pair and the fourth curved link pair is made from a polymer comprising polyethylene, polystyrene, nylon, polypropylene, polyvinylchloride, neoprene, cellulose, rubber, silicone, tetrafluoroethylene, polyamide, polybutadiene, and copolymers thereof.
